(19) United States
(12) Patent Application Publication
(10) Pub. No.: US 2007/0060834 A1
(43) Pub. Date: Mar. 15, 2007

(54) Catheter control console

(76) Inventors: Rick Matthew Odland, Roseville, MN (US); James D. Stice, Dayton, MN (US); Scott R. Wilson, Maple Grove, MN (US)

Correspondence Address: Richard C. Emery, 4189 Lakewood Avenue, White Bear Lake, MN, 55110-3925, US

(21) Appl. No.: 11/210,072

(22) Filed: Aug. 23, 2005

(51) Int. Cl. A61B 5/00

(52) U.S. Cl. 600/561; 600/300

(57) ABSTRACT

The invention is directed to a console coupleable to an indwelling catheter for monitoring interstitial pressure, collecting and withdrawing excess interstitial fluid and infusing fluids into the treated area.

### FIELD OF THE INVENTION

The invention relates to a device coupleable with an indwelling catheter used in site specific therapy which combines the functions of data display and data storage and also contains a vacuum pump for withdrawal of collected bodily fluid. In one embodiment, the invention further contains an infusion pump.

### BACKGROUND

A number of clinical conditions involve (e.g., are caused by and/or themselves cause) impaired circulation, and particularly circulation within interstitial spaces and within discrete, localized tissues. Among the more difficult examples of circulatory problems are those that involve localized tissue swelling, including compartment syndrome and edema (particularly cerebral edema), as well as solid and semisolid tumors forming confined sites within the body.

Acute compartment syndrome (ACS) generally involves impaired circulation within an enclosed fascial space (e.g., the anterior compartment of the lower leg), leading to increased tissue pressure and necrosis of muscle and nerves. The soft tissue of the lower leg is contained within four compartments, each bounded by heavy fascia: the anterior, lateral, superficial posterior, and deep posterior compartments. Once diagnosed, the injury requires immediate decompression through surgical release of the skin and fascia covering the area. Other proposed treatment strategies include the use of a sympathetic blockage, hyperbaric oxygen therapy and treatment with mannitol and allopurinol.

Several different apparatus and methods have been proposed for treating ACS. Among these are drainage by the use of catheters and similar instruments inserted into tissue sites including syringes, breast pumps, dental suction devices and waste gas scavenging. See, for example, the Mini VAC (Vacuum Assisted Closure) device, available from KCl (San Antonio, Tex.). The Mini VAC device provides negative pressure therapy for treating chronic and acute wounds and allows for the measurement and displaying of therapy at the wound site through micro-processor control and multi-lumen tubing. Negative pressure is applied to a special dressing positioned in a wound cavity or over a flap or graft. The pressure distributing wound dressing supposedly helps remove fluids from the wound. It should be mentioned that the Mini Vac is not used to prevent ACS but rather to treat the wound from the fasciotomy to decompress ACS.

A “mechanical leech” has been developed, which attaches to a wound site to remove blood and promote wound healing. See, the University of Wisconsin Press release dated Dec. 12, 2001, “Novel Device Takes Over Where Medicinal Leeches Leave Off.”

See also U.S. Pat. No. 5,484,399, which describes a method and apparatus for reducing interstitial fluid pressure in tissues, particularly in tumors, by applying suction to the interior of the tissue. The method comprises inserting into the tissue one or more needle-like, elongated tubes, each having at least one hole at or near the end that is inserted into the tissue and each having means to apply suction to the protruding end. Components may be provided to measure the pressure within the tissue and to use this measurement to control the suction applied to the tissue through the tubes.

Applicant has also previously described methods and related systems for use in site specific therapy of a tissue site. See issued U.S. Pat. Nos. 6,030,358, 6,537,241 and published PCT application PCT/US98/16416, the disclosures of which are all incorporated herein by reference. In one embodiment, the PCT application provides a system that comprises one or more catheters adapted to be positioned within the tissue site and a delivery/recovery mechanism for employing the catheter(s) to control the movement of bulk fluids and/or active fluid components within or between tissue portions or adjacent tissues in a manner that achieves a therapeutic effect. The catheters, in turn, can comprise one or more semipermeable microcatheters, adapted to effect the movement of fluid or fluid components within the tissue site by microdialysis within the tissue site. In its various embodiments, the system previously described by Applicant can be used for the treatment of a variety of disorders, including cerebral edema and compartment syndrome.

Bioactive agents, such as drugs, have long been delivered to the body by a wide variety of methods, including ingestion, inhalation, injection and transdermally, among others. With any remote or systemic delivery the ability of the delivered drug to access its intended site of action is often dependent on a number or parameters, including: (1) the flow kinetics of fluids containing such drugs, such as blood, into or through the desired site; (2) the concentration and form of drugs within those fluids; and (3) the extent to which the drug is able to be transferred to, or concentrated within, the desired site from the fluid. As compared with systemic delivery, site specific approaches for delivering drugs have been developed as well and include the use of indwelling catheters and implantable drug delivery devices.

The delivery of drugs to tumors, however, presents additional challenges beyond those associated with both systemic and site specific delivery techniques. Reduced perfusion to tumors is known to limit the ability to effectively deliver chemotherapeutic agents to the tumor. One of the most pervasive and compelling theories regarding causation is the Baxter-Jain hypothesis, which states that elevated tissue pressure within the tumor limits tumor perfusion. The theory and modeling suggest that absence of lymphatics plays a key role in pathogenesis of the interstitial hypertension.

A variety of approaches have been considered to improve the delivery of drugs to tumors. For instance infusion of fluid directly into tissues bypasses the microcirculatory system and has been attempted with mixed results. Investigators have shown, for instance, that fluid injected too rapidly into tumors caused a shearing action that results in an inhomogeneous distribution.

More recently, improved perfusion by the removal of excess interstitial fluid has been proposed as well. See the papers and patents or applications of DiResta et al., including Ann. biomed. Eng. 28:543-555 (2000) and Ann. Biomed Eng. 28:556-564 (2000), as well as U.S. Pat. No. 5,484,399 (“Process and Device to Reduce Interstitial Fluid Pressure in Tissue”), and U.S. Patent Pub. US2001/0047152 (“Apparatus and Method for Reducing Interstitial Fluid Pressure and Enhancing Delivery of a Therapeutic Agent”).

DiResta et al. suggest a therapeutic effect can be derived by the alleviation of interstitial hypertension and have developed an “artificial lymphatic system” or “ALS”. These various references establish that removal of fluid from tumors increase blood flow, oxygenation and delivery of a dye to the hypoperfused center. In these studies, an “aspiration probe”, in the form of a stainless steel needle, was used to remove tissue fluid. DiResta et al. also demonstrated that fluid removal will result in shrinkage of the tumor compared with controls when chemotherapeutic drug treatment is given systemically. The device consists of a needle with slits along its length that is inserted into the center of the tumor through which suction is applied. Treatment with ALS has reduced interstitial hypertension, increased tumor blood flow, and enhanced dye penetration into the tumor. When used with intravenous chemotherapy, ALS has resulted in significant reduction in tumor growth.

U.S. Patent Publication 2001/0047152 (DiResta et al.) further describes the manner in which cancer therapy may be categorized into three major approaches: (1) Surgical Excision; (2) Radiotherapy; and (3) Chemotherapy. Chemotherapy is defined as the treatment of cancer by a systemic administration of drugs. Unfortunately, most drugs which showed promising effects in vitro have failed to be as effective in vivo, particularly in solid tumors. It has been suggested that one of the major reasons for this failure is the impediment of drug transport into tumors. In particular, a physiological barrier created by raised interstitial fluid pressure appears to be responsible the interstitial fluid pressure is raised in tumors primarily because of the lack of lymphatics in tumors and growth of tumor cells in confined spaces. The raised interstitial fluid pressure in tumors is a principal transport-retarding factor for the delivery of drugs such as macromolecules, i.e., large molecular weight molecules such as monoclonal antibodies (MoAb), tumor necrosis factor and other chemotherapeutic agents.

The above-cited DiResta et al. application goes on to describe an interstitial fluid pressure reducing apparatus that includes an aspiration probe having a body with an open proximal end and a closed distal end, an interior chamber defined by the body and proximal and distal ends, and at least one slit along the body providing fluid communication between the chamber of the aspiration probe and tissue upon insertion of at least a portion of the aspiration probe in tissue. The proximal end is configured and dimensioned for coupling to a suction source for generating suction in the chamber of the aspiration probe upon connection with the aspiration probe to thereby reduce interstitial fluid pressure of the tissue.

Despite these promising findings, the above-cited DiResta articles each describe various “limitations” to their use of ALS, including apparent limitations regarding the extent of vacuum that can be safely applied to the aspiration probe, and the concern that drugs may be removed along with the fluid itself. The authors describe the range of about −50 to −80 mm Hg as being an optimal vacuum range under their experimental conditions. In addition, the small holes of the ALS could potentially be problematic. As a result, long term use of the ALS may be further complicated by the lack of a mechanism to secure the drains of the ALS to the tissue so that the drains remain spatially fixed. Finally, the ALS had no features that facilitate use with adjunct therapies that enhance drug delivery. Use of various other therapies to enhance efficacy of the ALS would be desirable. However, until now, no other adjunct therapy has been used in conjunction with the ALS.

In an effort to address interstitial hypertension within the body, including that associated with tissue swelling and tumor formation, Applicant has developed systems, methods and apparatus allowing integrated pressure monitoring, aspiration and fluid infusion (See U.S. Pat. Nos. 6,030,358, 6,537,241, U.S. Patent Publications 2003-0167031, 2003-0187367, Ser. No. 10/508,610, and PCT/US03/08921, the disclosures of which are herein incorporated by reference. While systems, methods and apparatus have been developed to monitor pressure, aspirate and infuse fluids, separate pieces of equipment are presently used to achieve each of these functions. What is clearly needed is, therefore, a single unit attachable to the indwelling pressure monitoring, infusing and fluid collection catheter that is able to interpret and monitor pressure signals, remove water and other fluids and also infuse fluids into the treatment site.

### SUMMARY

In one aspect, the invention comprises a console for use with an indwelling pressure monitoring, infusing and fluid collection catheter, having a power supply for supplying uniform and controlled energy. A vacuum pump is coupleable to the catheter for extraction of bodily fluids collected by the catheter and an infusion pump is coupleable to the catheter for infusion of fluids. A pressure signal conditioner is in communication with a pressure transducer coupled to the catheter for converting analog pressure signals generated by the pressure transducer to digital signals. A microprocessor control unit is in communication with the vacuum pump controlling extraction of bodily fluids through the catheter and in communication with the infusion pump for infusing fluids through the catheter and in communication with the pressure transducer for processing received digital pressure signals. A display is also part of the console for displaying parameters and processed pressure signals.

In another aspect, the invention comprises a console for use with an indwelling pressure monitoring, infusing and fluid collection catheter, the catheter having a pressure tube, an outer tube defining a lumen and hollow fibers in fluid communication with the lumen of the outer tube. A vacuum pump is coupleable to the outer lumen of the catheter for extraction of bodily fluids collected by the catheter and an infusion pump is coupleable with the pressure tube of the catheter for infusion of fluids collected by the hollow fibers. A pressure signal conditioner is in communication with a pressure transducer coupled to the pressure tube for converting analog pressure signals generated by the pressure transducer to digital pressure signals. A microprocessor control unit is in communication with the vacuum pump for controlling extraction of bodily fluids according to parameters and in communication with the infusion pump for controlling fluid infusion according to parameters and processing received pressure signals. A user interface is in communication with the microprocessor control unit for setting parameters; and a display is in communication with the microprocessor control unit for displaying set parameters and processed pressure signals.

In yet another aspect, the invention comprises a console for use with an indwelling pressure monitoring, infusion and fluid collection catheter, the catheter having a pressure tube, an outer tube defining a lumen and hollow fibers in fluid communication with the lumen of the outer tube comprising. A vacuum pump is coupleable to the outer lumen of the catheter for extraction of bodily fluids collected by the catheter and an infusion pump is coupleable with the pressure tube of the catheter for infusion of fluids collected by the hollow fibers. The console has a power section, comprising a power supply attachable to a power source, a battery charger attached to the power supply and a battery attached to the battery charger. Also included within the power section is a power switch over attached to the power supply and the battery for providing battery power when the power source fails. A battery charge monitor is in communication with the battery and a microprocessor control unit to monitor the battery to determine when battery voltage drops below a predetermined value and allow the battery charger to charge the battery and to prevent overcharging of the battery. A power conditioning power supply for suppressing electrical surges and supplying a uniform power stream to the microprocessor.

The console has a digital section, comprising the microprocessor control unit connected to the power switch over and to the battery charger for controlling battery charging. The microprocessor control unit is in communication with the vacuum pump for controlling extraction of bodily fluids according to parameters and in communication with the infusion pump for controlling fluid infusion according to parameters and processing received pressure signals. A display is in communication with the microprocessor control unit for displaying set parameters and processed pressure signals. A user interface is in communication with the microprocessor control unit for communicating with the microprocessor control unit and for setting parameters and an NV RAM unit is in communication with the microprocessor control unit for storing the operating system and software. An infusion pump control is in communication with the microprocessor control unit and the infusion pump to control the rate of infusion and whether the infusion pump is on or off. Likewise, a vacuum pump control is in communication with the microprocessor control unit and the vacuum pump to turn the vacuum pump on and off. A real time clock is in communication with the microprocessor control unit to record the timing of the medical procedure and a USB interface is in communication with the microprocessor control unit to allow for downloading or transfer of generated data.

The console also has an analog section comprising a pressure signal conditioner in communication with a pressure transducer coupled to the pressure tube for converting analog pressure signals generated by the pressure transducer to digital pressure signals. An isolation transformer is connected between the microprocessor and the pressure signal conditioner to absorb excess voltage.

In an alternative aspect, the invention comprises console for use with an indwelling pressure monitoring/fluid collection catheter. The console has a power supply for supplying uniform and controlled energy and a vacuum pump coupleable to the catheter for extraction of bodily fluids collected by the catheter. A pressure signal conditioner is in communication with a pressure transducer embedded within the catheter for converting analog pressure signals generated by the pressure transducer to digital signals. A microprocessor control unit is in communication with the vacuum pump for controlling extraction of bodily fluids through the catheter and with the pressure transducer for processing received digital pressure signals. A display is in communication with the microprocessor control unit for displaying parameters and processed pressure signals.

These and other features, aspects and advantages of the present invention will become better understood with reference to the following description and appended claims.

### BRIEF DESCRIPTION OF THE DRAWINGS

FIG. 1 is a plan view of an indwelling pressure displaying and fluid collection catheter system.

FIG. 2 is a detailed view of the catheter tip shown in FIG. 1.

FIG. 2a is a longitudinal section of the catheter taken at the point that the hollow fibers emerge from the outer tube as designated by brackets 2a-2a.

FIG. 3 is a cross section taken through lines 3-3 of the catheter shown in FIG. 1.

FIG. 3a is a cross section taken through lines 3a-3a of the catheter shown in FIG. 1.

FIG. 4 is a block diagram of an embodiment of a displaying, collection and infusion console.

FIG. 5 is a module block diagram of an embodiment of the console.

FIG. 6 shows an exterior perspective view of the housing.

FIG. 7 is a block diagram of an embodiment of the console.

FIG. 8 is a block diagram of an embodiment of the console.

### DETAILED DESCRIPTION

### Nomenclature

10 Indwelling Pressure Monitoring, Infusing and Fluid Collection Catheter System
12 Catheter Body
14 First Y Connector
14a First Branch of First Y Connector
14b Second Branch of First Y Connector
16 Pressure Line
18 Pressure Transducer
19 Vent Line
20 Second Y Connector
20a First Branch of Second Y Connector
20b Second Branch of Second Y Connector
22 Vent
22a Valve
23 Infusion Line
24 Third Y Connector
24a First Branch of Third Y Connector
24b Second Branch of Third Y Connector
26 Fluid Collection Port
28 Vacuum Line
30 Vacuum Connector
32 Pressure Signal Connection
100 Slit Pressure Lumen
101 Slit
102 Tip Section of Catheter Body
104 Hollow Fiber
105 Hollow Fiber Lumen
106 Outer Tube
108 First Lumen
110 Inner Tube
112 Second Lumen
114 First Adhesive Joint
116 Second Adhesive Joint
200 Monitoring, Infusion and Collection Console
202 Display
204 Vacuum Pump
206 Infusion Pump
208 Mounting Platform
210 Microprocessor
212 Real Time Clock
214 USB Interface
216 Vacuum Pump Control
218 Battery Charge Monitor
220 Keypad
222 NV RAM
224 Battery
226 Battery Charger
228 Power Supply
230 Power Switch Over
232 Power Conditioning Power Supplies
234 Infusion Pump Control
236 Memory Stick
238 Housing
240 Power Section
242 Digital Section
244 Analog Section
246 Pressure Signal Port
248 Power Switch
250 Vacuum Port
252 Infusion Port
254 Pressure Signal Conditioner
256 Isolation Transformer
300 Monitoring and Collection Console

### Definitions

“Coupleable” means capable of being coupled to another structure.

“CRT” refers to a cathode ray tube.

“LCD” refers to a liquid crystal display.

“Mounting Platform” refers to any structure used to mount the components of the invention on.

### Construction

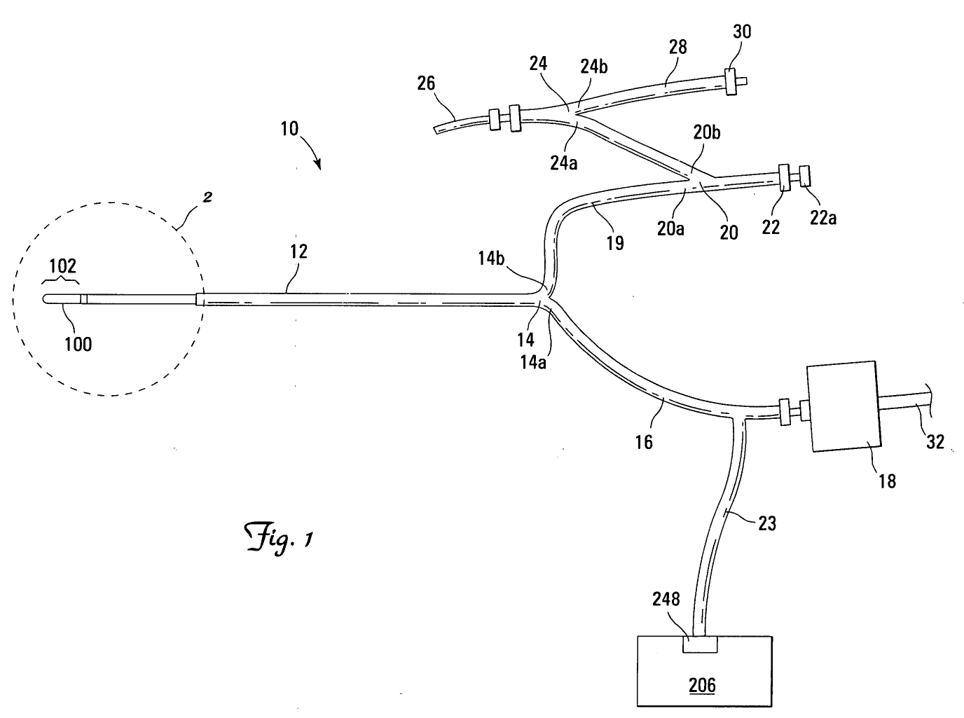

FIG. 1 is a plan view of an indwelling pressure monitoring, infusing and fluid collection catheter system 10. The catheter system comprises a catheter body 12, which is attached to and in fluid communication with a first branch 14a of a first Y connector 14 to a pressure line 16. An infusion line 23 is attached to and in fluid communication with the pressure line 16 at its distal end (unnumbered) and at its proximal end (unnumbered) is attached to and in fluid communication with an infusion pump 206. A pressure transducer 18 terminates the pressure line 16. A pressure signal connection 32 extends from the pressure transducer 18 and attaches to the console 200 as explained below in greater detail. It should be mentioned that in one embodiment the pressure signal connection 32 as specified is an electrical connection, other connections such as fiber optic (not shown) or wireless (not shown) are also possible and therefore within the scope of the invention. The second branch 14b of the first Y connector 14 is attached to and in fluid communication with a first branch 20a of a second Y connector 20 which itself is attached to and in fluid communication with a vent 22. The vent 22 is terminated by a valve 22a and is used to drain any fluid from the vent line 19 prior to beginning the procedure. With the vacuum pump 204 running, if the valve 22a is opened, any fluid in the line is drained into a collection chamber (not shown) and can be used for analysis. The second branch 20b of the second Y connector 20 is attached to and fluid communication with a third Y connector 24, which further defines a first branch 24a which is attached to and in fluid communication with a fluid collection port 26. A second branch 24b of the third Y connector 24 is attached to and in fluid communication with a vacuum line 28, which terminates at vacuum connector 30.

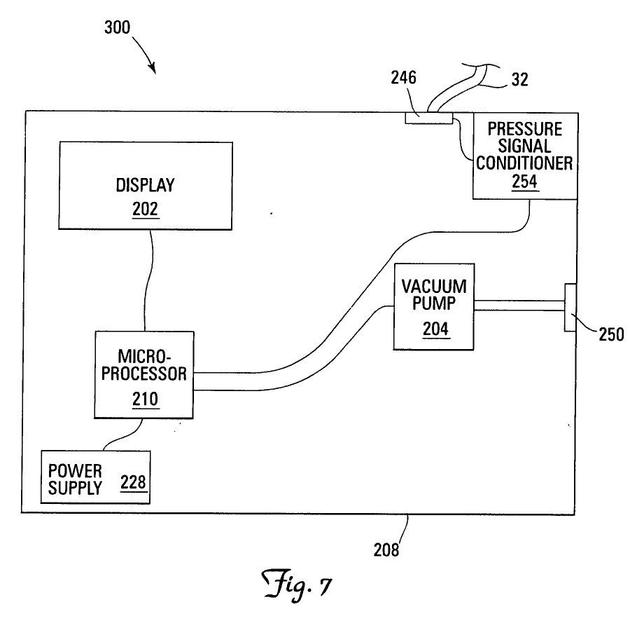

In another embodiment, a catheter (not shown) similar to the catheter 10 embodiment shown in FIG. 1 is contemplated by and therefore within the scope of the invention. The alternative catheter (not shown) differs from the catheter 10 in that the pressure transducer (not shown) is embedded in the distal tip (not shown) and sends generated signals via electrical communication to the console 300 as shown in FIG. 7. Because the pressure transducer (not shown) is embedded in the distal tip (not shown) of the catheter (not shown), there is no need to pressurize an inner tube 110 via an infusion pump 206, thus an infusion pump 206 is eliminated from the console 300.

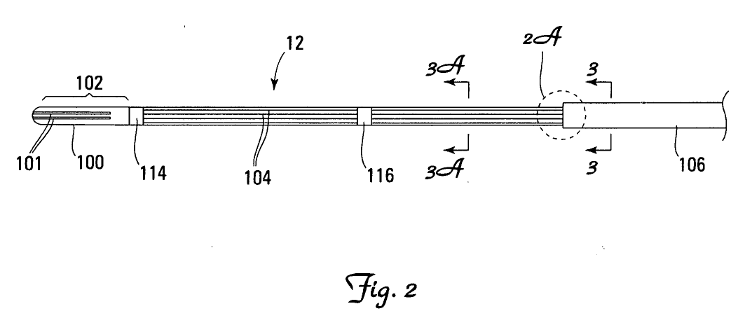

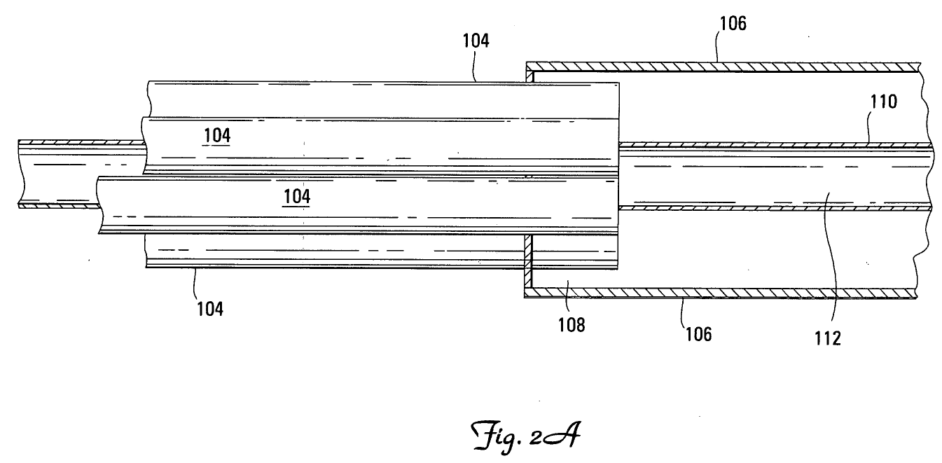

FIG. 2 is a plan view of the distal section (unnumbered) of the catheter body 12. The tip section 102 comprises a slit pressure lumen 100 further defining at least one slit 101 which is attached to and in fluid communication with the distal end (unnumbered) of the catheter body 12. The slit pressure lumen 100 extends distally past the point where the hollow fibers 104 terminate and serves to protect an inner tube 110 which extends the length of the catheter body 12 and distally terminates within the space defined by the slit pressure lumen 100. Hollow fibers 104 are held together by first 114 and second 116 adhesive joints. The inner tube 110 further defines a second lumen 112 and functions to measure pressure in the affected anatomical region by being primed with a saline solution by means of an infusion pump 206 which is attached to and in fluid communication with the inner tube 110 by infusion line 23, prior to and during insertion of the catheter body 12 into the patient. The inner tube 110 is also attached to and in fluid communication with a pressure transducer 18. The pressure transducer 18 responds to changes in pressure measured by the inner tube 110 following introduction into a patient by generating electrical signals which are transmitted to the microprocessor by an electrical connection 32. Attached to and in communication with the pressure transducer 18 is a pressure signal conditioner 254 which functions to convert electrical signals generated by the pressure transducer 18 into corresponding digital signals that can be received and processed by the microprocessor 210. In order to maintain patency through the slit pressure lumen 100, a constant flow of saline solution is maintained throughout the treatment procedure through the second lumen 112 by the infusion pump 206 via infusion line 23, which is attached to the console 200 by means of the infusion port 250 as discussed in greater detail below. It has been found that a saline infusion rate of approximately 511/hour adequately keeps the area around the termination of the inner tube 110 free of any clotting or other biological debris that might result in an inaccurate pressure reading. In one embodiment (not shown) a plurality of infusion pumps 206 is incorporated into the invention to provide for simultaneous infusion of other fluids such as chemotherapeutic drugs directly to a treatment site.

FIG. 2a is a lateral section of the portion of the catheter body 12 where the hollow fibers 104 terminate inside the first lumen 108 of the outer tube 106.

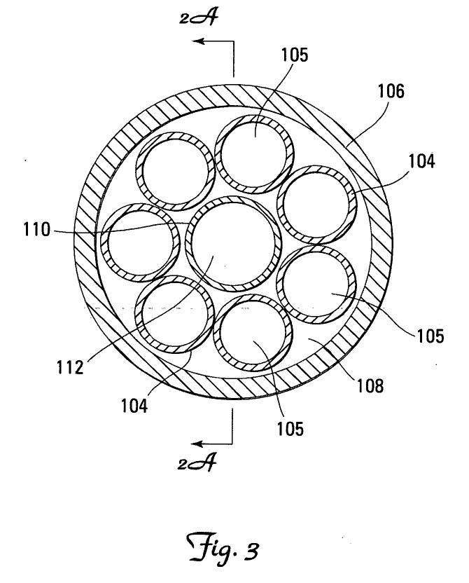

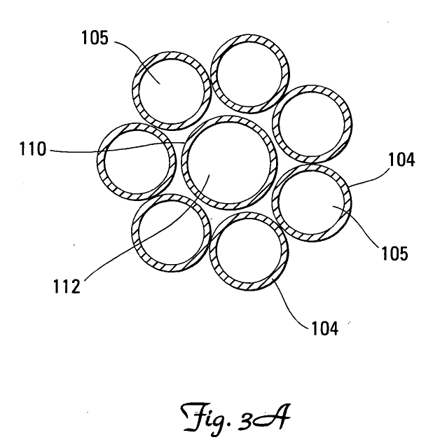

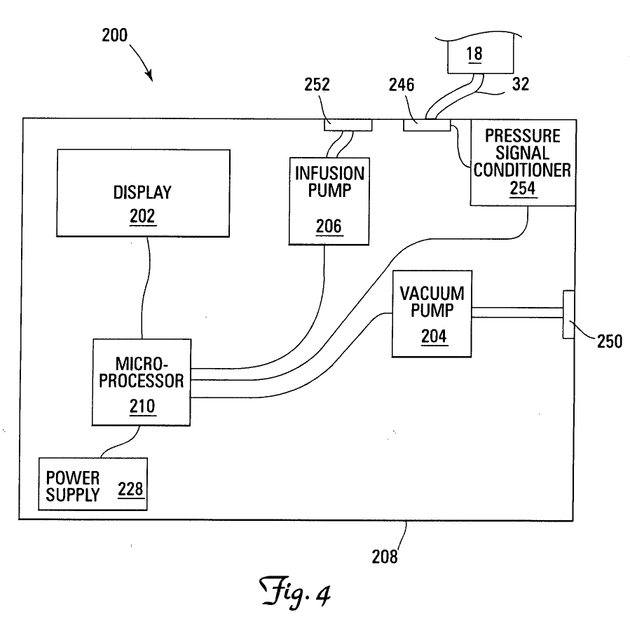

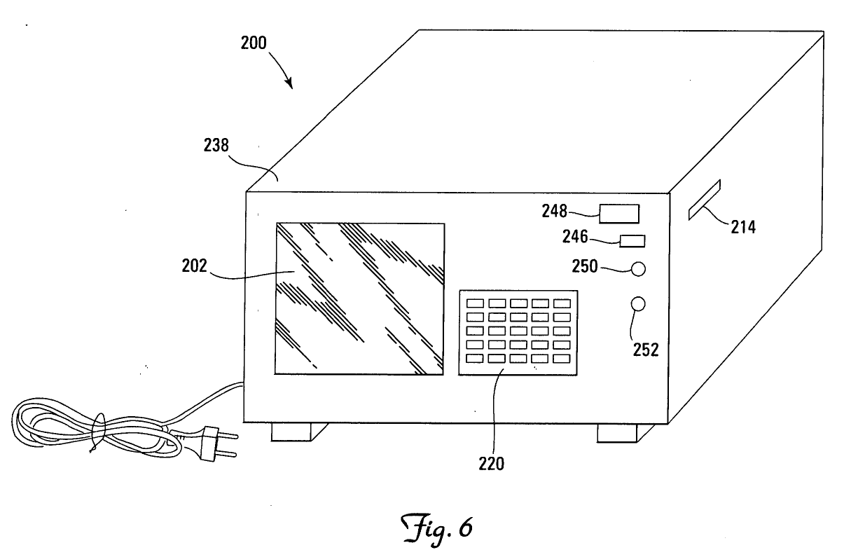

FIG. 3 is a cross sectional view of the catheter body 12 taken between the lines 3-3. An outer tube 106 made of polyimide defines a first lumen 108 which is in fluid communication with a lumen 105 of a hollow fiber 104 to provide a pathway for the collection and delivery of interstitial fluid during treatment. Polyimide is used because of its well known use in other blood contact devices, its resistance to kinking and its inherent mechanical strength in thin wall applications. Extending the length of the catheter body 12, pressure line 16 and located within the first lumen 108 and coaxial with the outer tube 106 is the inner tube 110, which defines a second lumen 112. Extending distally from the first lumen 108 and terminating proximally within the first lumen 108 is at least one and preferably a plurality of hollow fibers 104. Each hollow fiber 104 is a 0.1 micron pore size polysulfone hollow tube, with an outer diameter of approximately 360 microns and an inner diameter of approximately 280 microns by 8 cm long. While specific dimensions are given here, it should be understood that it is for the purpose of illustration only and should not be construed as limiting the scope of the invention. The hollow fiber(s) 104 define a distal end (unnumbered) which is sealed, and an open proximal end (unnumbered) which is connected to and in fluid communication with the first lumen 108. As interstitial fluid contacts the outer surfaces (unnumbered) of the hollow fiber(s) 104 it passes through the wall (unnumbered) of the hollow fiber 104 to enter the hollow fiber lumen 105 and eventually contacts the first lumen 108 within the outer tube 106 as a vacuum is drawn through the first lumen 108. At the proximal end (unnumbered) of the catheter body 12, the first lumen 108 is connected to and in fluid communication with a vent line 19 connected to the vent 22 for air expulsion, the fluid collection port 26 for fluid collection access and finally a vacuum line 28 to provide connection to a vacuum pump 204 as best shown in FIG. 4 and FIG. 6. FIG. 3a is a cross section taken through the lines 3a-3a in FIG. 2 and shows the same components minus the outer tube 106.

FIG. 4 shows a block diagram of the components of the monitoring, collection and infusion console 200. A microprocessor control unit 210 is in communication with a display 202, a vacuum pump 204, an infusion pump 206 and the pressure signal conditioner 254 which receives and converts to digital, analog signals generated by the pressure transducer 18. A power supply 228 which provides a steady source of electric power having the desired characteristics is connected to a power source which can be either 110V or 220V. In a preferred embodiment all connections between the microprocessor control unit 210, display 202, vacuum pump 204, infusion pump and a keypad 220 are electrical, however other types of connections such as fiber optic and wireless are also contemplated by and therefore within the scope of the invention. The pressure transducer 18 generates electrical signals that are received by the microprocessor control unit 210 via electrical connection 32 through pressure signal port 246. The vacuum pump is attached to and in fluid communication with the first lumen 108 through the vacuum port 250. The infusion pump 206 is attached to and in fluid communication with the second lumen 112 through the infusion port 252. The infusion pump 206 can be a syringe pump (not shown) which is of conventional design and well known to those having ordinary skill in the art. In an alternative embodiment, the infusion pump 206 could also be a fluid impermeable sac containing fluid which is pressurized by applying external mechanical pressure on the sac. The various components are mounted to mounting platform 208.

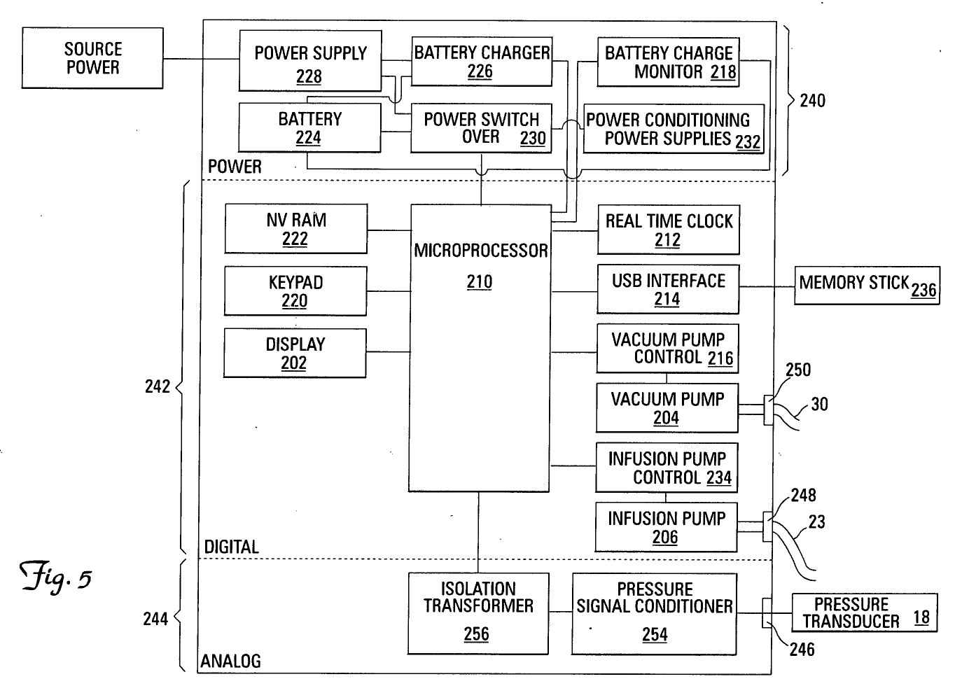

FIG. 5 shows a detailed electrical block diagram showing the electrical components and the connections between them. The various components are mounted to a mounting platform 208, such as a housing 238 as best shown in FIG. 6. Three sections are defined in the diagram: (1) power section 240 which distributes and conditions the amount of electrical energy going to the various electrically powered components; (2) a digital section 242 which contains the digitally operating components; and (3) an analog section 244 which contains the analogue operating components. Within the power section 240 are a power supply 228 which is externally connected to either a 110V or 220V power source (not shown) and internally attached to a power switch over 230. The power supply 228 is also electrically connected to a battery charger 226 which is in turn electrically connected to a rechargeable battery 224. The battery 224 is also connected to the power switch over 230 whose function is to supply power to a microprocessor 210. In the event of an external power failure, the power switch over 230 would automatically and instantly switch the internal power to the battery 224 to prevent interruption of treatment. A power conditioning power supply 232 is also attached to the power switch over 230 and functions as a surge suppressor as well as supplying a uniform power stream to the microprocessor 210. A battery charge monitor 218 is electrically connected to the microprocessor 210 and electrically connected to the battery 224. The battery charge monitor 218 is controlled by the microprocessor 210 and functions to charge the battery 224 when the microprocessor 210, as programmed by the software, senses charging is necessary.

The microprocessor 210 is separately and electrically connected to the various components of the digital section: an NV RAM 222 is electrically and directly connected to the microprocessor 210 which stores the operating system, software and collected data; keypad 220 is electrically and directly connected to the microprocessor 210 which allows the console operator to access the various functions provided by the console 200 and input information as required; a display 202 is electrically and directly connected to the microprocessor 210 which can be a CRT, LCD, LED or other digital or analog display type and displays the various functions and readings made by the indwelling pressure displaying, infusing and fluid collection catheter system 10; a real time clock 212 is electrically and directly connected directly to the microprocessor 210 to eventually display and record the timing of the medical procedure. It should be mentioned that a display 202 could also incorporate touch screen technology (not shown) wherein the keypad 220 appears as part of the display and control is achieved by touching the appropriate area of the screen. A vacuum pump control 216 is electrically and directly connected to the microprocessor 210 and in turn electrically and directly connected to a vacuum pump 204. The vacuum pump control 216 functions to turn the vacuum pump 204 on and off as well as regulate the amount of vacuum produced, however, the vacuum pump control 216 is optional and the console 200 would also function using a continuous vacuum. In another embodiment, a mechanical valve (not shown) is manually opened or closed by the operator as deemed necessary for treatment.

An infusion pump control 234 is electrically connected to the microprocessor 210 and electrically connected to the infusion pump 206 and functions to control the rate of infusion as well as whether the infusion pump 206 is on or off. In one embodiment the infusion pump 206 is a simple syringe pump (not shown) of conventional design and well known to those having ordinary skill in the art, in which a loaded syringe (not shown) is loaded into a mechanism which depressed the syringe plunger, thus infusing the loaded fluid. A suitable syringe pump (not shown) is model NE-500, manufactured by New Era Pump Systems, Farmingdale, N.Y. In another embodiment (not shown) the infusion pump 206 comprises a fluid impermeable sac containing fluid which is pressurized by applying external mechanical pressure on the sac.

A USB interface 214 is electrically connected to the microprocessor 210 and functions as an attachment point for a memory stick 236 to download and save data for future use or analysis.

In the analog section 244 a pressure signal conditioner 254 is coupleable with the pressure transducer 18 which is discussed in detail above. The pressure signal conditioner 254 amplifies and filters out electronic noise in electrical signals generated by the pressure transducer 18 and is electrically connected with the isolation transformer 256. The isolation transformer 256 is in communication with the microprocessor 210 and functions to absorb excess voltage and also to isolate the line voltage from the patient. A suitable isolation transformer is part number 9399-V2-00, manufactured by Piltron, Toronto, Ontario.

In operation the indwelling pressure displaying, infusing and fluid collection catheter system 10 is connected to the displaying, infusion and collection console 200 by connecting the electrical connection 32 (emanating from the pressure transducer 18) to a pressure signal port 246. The pressure transducer 18 is connected to the pressure line 16 which is connected to the first branch 14a of the first Y connector 14, which is connected to the second lumen 112 of the inner tube 110. The infusion line 23 is connected to the infusion pump 206 via an infusion pump port 248 on its proximal end and to the pressure line 16 on its distal end. The vacuum pump 204 is connected to the first lumen 108 by vacuum line 28 via vacuum connector 30.

The invention does not show, but contemplates and therefore encompasses in scope, an input (not shown) for a blood pressure sensor (not shown). A blood pressure sensor (not shown), integrated with the catheter (not shown) or independent (not shown) would generated blood pressure signals which would be read by the console. Blood perfusion pressure (the difference between blood pressure and tissue pressure) would be calculated, displayed and stored by the console.

FIG. 6 shows the console 200 enclosed in a housing 238.

FIG. 7 shows an alternative embodiment of the console 300 which is similar to the embodiment shown in FIG. 4 with the exclusion of the infusion pump 206 and infusion port 252. This embodiment of the console 300 is attached to a catheter (not shown) which has a pressure transducer (not shown) embedded in the distal tip of the catheter, which does not require infusion of saline solution during treatment.

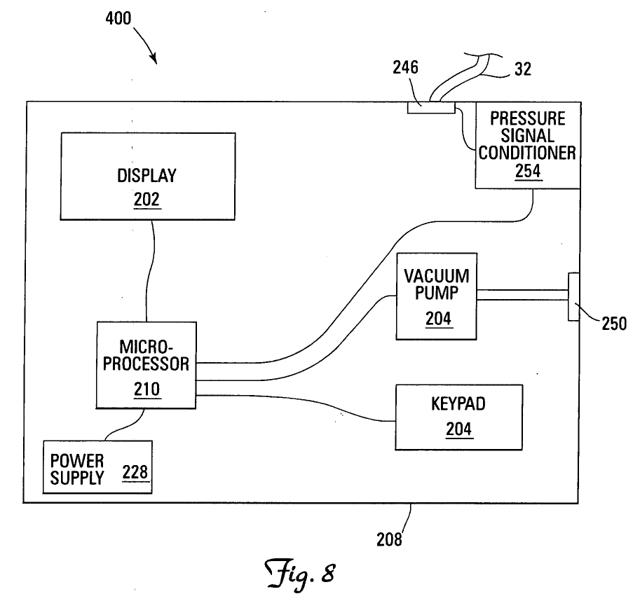

FIG. 8 shows yet another alternative embodiment of the console 400 which is similar to the embodiment shown in FIG. 4, but with the addition of a user interface in the form of a keypad 220. The keypad 220 is electrically and directly connected to the microprocessor 210 which allows the console operator to access the various functions provided by the console 200 and input information as required.

### Use

Using the present invention involves inserting the indwelling pressure displaying and fluid collection catheter 10 into the targeted muscle compartment by skin puncture via an introducer (not shown). The second lumen 112 is infused with saline solution prior to insertion and connected to the pressure transducer 18 which generates and sends analog electrical signals to the microprocessor 210 via the pressure signal conditioner 254, which are eventually displayed on the display 202. Interstitial fluid pressure is measured at the tip 102 of the catheter body 12 through the slit pressure lumen 100. Fluid within the second lumen 112 is displaced corresponding with the amount of pressure within the muscle compartment or tumor and the pressure transducer 18 generates a signal which is transmitted to the display 202 where a reading can be manually taken or digitally recorded. Catheter 12 patency is maintained by the slit pressure lumen 100 and by continuous infusion of a very small volume of sterile normal saline (approximately 5 μL/hr) by the infusion pump 206 which is connected to and in fluid communication with the second lumen 112.

The first lumen 108 is typically maintained under cyclic on/off vacuum (−50 mm Hg for 3 minutes, 0 mm Hg for 3 minutes) by connection with the vacuum pump 204. During the vacuum phase interstitial fluid passes through the hollow fibers 104 and moved from the first lumen 108 within catheter 12 eventually to the fluid collection port 26, where it is collected. Fluid samples can be taken from the reservoir when desired using a standard syringe. During the off phase, interstitial fluid is replenished in the region near the catheter tip 102 by the hydraulic pressure gradient induced during the vacuum phase. Pressure readings are observed on the display 202 and treatment adjusted as necessary.

The immediate effect of using the console 200, 300 when attached to a catheter system 100, 400 is that the removal of fluid reduced the interstitial pressure or at least prevents the pressure from increasing. The pressure monitoring function is used to verify that interstitial pressure has been reduced or not increased. Fluid removed during the procedure can then be used for analysis to aid in determining further treatment. In the case of Compartment syndrome, it is possible that the analysis will determine if the patient is developing Compartment Syndrome, wherein additional measures can be taken. In other applications, such as tumors, additional diagnostics can be done to determine further treatment.

Although the present invention has been described in considerable detail with reference to certain preferred versions thereof, other versions are possible. Therefore, the sprit and scope of the appended claims should not be limited to the description of the preferred versions contained herein.

1. A console for use with an indwelling pressure monitoring, infusing and fluid collection catheter, comprising:
a. a power supply for supplying uniform and controlled energy;
b. a vacuum pump;
c. an infusion pump:
d. a signal receiving input in communication with signal conditioner for converting analog signals to digital signals;
e. a microprocessor control unit in communication with the vacuum pump and in communication with the infusion pump and in communication with the signal receiving input for processing the digital pressure signals; and
f. a display in communication with the microprocessor for displaying parameters and processed pressure signals.

2. The console of claim 1 further comprising a user interface in communication with the microprocessor control unit for setting parameters.

3. The console of claim 1 further comprising the vacuum pump being coupleable to the catheter so as to be in fluid communication with the catheter for extraction of bodily fluids collected by the catheter.

4. The console of claim 1 further comprising the infusion pump being coupleable to the catheter so as to be in fluid communication with the catheter for the infusion of fluids through the catheter.

5. The console of claim 1 further comprising the signal receiving input being capable of receiving signals generated by a pressure transducer.

6. The console of claim 5 wherein the signals received by the signal receiving input are electrical signals.

7. The console of claim 1 wherein the vacuum pump, infusion pump, signal receiving input, microprocessor, user interface and display are attached to a mounting platform.

8. The console of claim 7 wherein the mounting platform is a housing.

9. A console for use with an indwelling pressure monitoring, infusing and fluid collection catheter, the catheter having a pressure tube, an outer tube defining a lumen and hollow fibers in fluid communication with the lumen of the outer tube comprising:
a. a vacuum pump coupleable to of the catheter for extraction of bodily fluids collected by the catheter;
b. an infusion pump coupleable to the catheter for infusion of fluids collected by the hollow fibers;
c. a signal receiving input in communication with a pressure signal conditioner in communication with a pressure transducer coupled to the pressure tube for converting analog pressure signals generated by the pressure transducer to digital pressure signals;
d: a microprocessor control unit in communication with the vacuum pump for controlling extraction of bodily fluids according to parameters and in communication with the infusion pump for controlling fluid infusion according to parameters and processing received pressure signals;
e. a user interface in communication with the microprocessor control unit for setting parameters; and
f. a display for displaying set parameters and processed pressure signals.

10. The console of claim 9 further comprising the vacuum pump being coupleable to the outer lumen of the catheter.

11. The console of claim 9 further comprising the infusion pump being coupleable to the pressure tube of the catheter.

12. (canceled)

13. The console of claim 9 wherein the vacuum pump, infusion pump, signal receiving input, microprocessor, user interface and display are attached to a mounting platform.

14. The console of claim 13 wherein the mounting platform is a housing.

15. A console for use with an indwelling pressure monitoring, infusion and fluid collection catheter, the catheter having a pressure tube, an outer tube defining a lumen and hollow fibers in fluid communication with the lumen of the outer tube comprising:
a. a vacuum pump coupleable to the catheter for extraction of bodily fluids collected by the catheter;
b. an infusion pump coupleable to of the catheter for infusion of fluids collected by the hollow fibers;
c. a power section, comprising
i. a power supply attachable to a power source,
ii. a battery charger attached to the power supply,
iii. a battery attached to the battery charger,
iv. a power switch over attached to the power supply and the battery for providing battery power when the power source fails, and
v. a battery charge monitor in communication with the battery and a microprocessor control unit to monitor the battery to determine when battery voltage drops below a predetermined value and allow the battery charger to charge the battery and to prevent overcharging of the battery.
vi. a power conditioning power supply for suppressing electrical surges and supplying a uniform power stream to the microprocessor;
d. a digital section, comprising
i. the microprocessor control unit connected to the power switch over and to the battery charger for controlling battery charging, the microprocessor control unit in communication with the vacuum pump for controlling extraction of bodily fluids according to parameters and in communication with the infusion pump for controlling fluid infusion according to parameters and processing received pressure signals,
ii. a display in communication with the microprocessor control unit for displaying set parameters and processed pressure signals,
iii. a user interface in communication with the microprocessor control unit for communicating with the microprocessor control unit and for setting parameters,
iv. an NV RAM unit in communication with the microprocessor control unit for storing the operating system, software and generated data,
v. an infusion pump control in communication with the microprocessor control unit and the infusion pump to control the rate of infusion and whether the infusion pump is on or off,
vi. a vacuum pump control in communication with the microprocessor control unit and the vacuum pump to turn the vacuum pump on and off,
vii. a real time clock in communication with the microprocessor control unit to record the timing of the medical procedure, and
viii. a USB interface in communication with the microprocessor control unit; and
e. an analog section, comprising
i. a signal receiving input in communications with a pressure signal conditioner in communication with a pressure transducer coupled to the pressure tube for converting analog pressure signals generated by the pressure transducer to digital pressure signals;
ii. an isolation transformer connected between the microprocessor and the pressure signal conditioner to absorb excess voltage and to isolate the line voltage from the patient.

16. The console of claim 15 further comprising the vacuum pump being coupleable to the outer lumen of the catheter.

17. The console of claim 15 further comprising the infusion pump being coupleable to the pressure tube of the catheter.

18. (canceled)

19. The console of claim 15 wherein at least the vacuum pump, infusion pump, signal receiving input capable of being in communication with a pressure transducer, microprocessor, power supply battery charger, battery, power switch over, power conditioning power supplies, NV RAM, keypad, infusion pump control, USB interface, vacuum pump control, battery charge display and display are attached to a mounting platform.

20. The console of claim 19 wherein the mounting platform is a housing.

21. A console for use with an indwelling pressure monitoring/fluid collection catheter, comprising:
a. a power supply for supplying uniform and controlled energy;
b. a vacuum pump;
c. a signal receiving input in communication with a pressure signal conditioner for converting analog pressure signals generated by the pressure transducer to digital signals;
d. a microprocessor control unit in communication with the vacuum pump and in communication with the pressure transducer for processing received digital pressure signals; and
e. a display in communication with the microprocessor for displaying parameters and processed pressure signals.

22. The console of claim 21 further comprising a user interface in communication with the microprocessor control unit for setting parameters.

23. The console of claim 21 further comprising the vacuum pump being coupleable to the catheter so as to be in fluid communication with the catheter.

24. (canceled)

25. The console of claim 21 further comprising the signal receiving input being capable of receiving signals generated by a pressure transducer coupled to the catheter.

26. The console of claim 25 wherein the signals received by the signal receiving input are electrical signals.

27. The console of claim 21 wherein the vacuum pump, signal receiving input, microprocessor, user interface and display are attached to a mounting platform.

28. The console of claim 27 wherein the mounting platform is a housing.

29. A method for monitoring internal pressure of a patient and infusing fluids through an indwelling catheter introduced into a patient, the catheter connected to a catheter control console and capable of generating pressure signals and having a fluid infusion channel, the method comprising the steps of:
a. providing a source of uniform power to the catheter control console;
b. receiving pressure signals;
c. converting the pressure signals to a displayable form thereby monitoring the internal pressure of a patient and
d. activating an infusion pump within the console thereby causing an increase in fluid pressure.

30. The method of claim 29 wherein the infusion pump is coupled to the fluid infusion channel of the catheter.

31. The method of claim 29 wherein activating the infusion of fluids through the catheter is controllable to prevent fluid overload.

32. A method for monitoring internal pressure and infusing fluids through an indwelling catheter introduced into a patient, the catheter capable of generating pressure signals and having a fluid infusion channel, the method comprising the steps of:
a. providing a source of uniform power to the catheter control console;
b. introducing the catheter into a selected anatomical region of the patient;
c. connecting the catheter to the console;
d. receiving of the pressure signals by the console;
e. converting the pressure signals to a displayable form thereby monitoring the internal pressure of a patient in the anatomical region of the patient receiving the introduced catheter; and
f. infusing fluids through the infusion channel of the catheter by activating an infusion pump within the console.

33. The method of claim 31 wherein the infusing of fluids through the catheter is controllable to prevent fluid overload.

34. A method for monitoring internal pressure, withdrawing fluids and infusing fluids through an indwelling catheter introduced into a patient and connected to a catheter control console, the catheter capable of generating pressure signals, having a vacuum channel and a fluid infusion channel, the method comprising the steps of:
a. providing a source of uniform power to the catheter control console;
b. activating a vacuum pump within the console thereby withdrawing excess fluid;
c. receiving pressure signals;
d. converting the pressure signals to a displayable form thereby monitoring the internal pressure of a patient and
e. activating an infusion pump within the console thereby causing an increase in fluid pressure.

35. The method of claim 33 wherein the infusion pump is coupled to the fluid infusion channel of the catheter.

36. The method of claim 33 wherein the vacuum pump is coupled to the vacuum channel of the catheter.

37. The method of claim 33 wherein activating the infusion of fluids through the catheter is controllable to prevent fluid overload.

38. A method for monitoring internal pressure, withdrawing fluids and infusing fluids through an indwelling catheter introduced into a patient, the catheter capable of generating pressure signals having a vacuum channel and having a fluid infusion channel, the method comprising the steps of:
a. providing a source of uniform power to the catheter control console;
b. introducing the catheter into a selected region of the patient;
c. connecting the catheter to the console;
d. activating a vacuum pump within the console thereby withdrawing excess fluid;
e. receiving of the pressure signals by the console;
f. converting the pressure signals to a displayable form thereby monitoring the internal pressure of a patient in the anatomical region of the patient receiving the introduced catheter; and
g. infusing fluids through the infusion channel of the catheter by activating an infusion pump within the console.

39. The method of claim 38 wherein activating the infusion of fluids through the catheter is controllable to prevent fluid overload.

40. A console for use with an indwelling pressure monitoring, infusing and fluid collection catheter, comprising:
a. a power supply for supplying uniform and controlled energy;
c. an infusion pump;
d. a signal receiving input in communication with a signal conditioner for converting analog signals to digital signals;
e. a microprocessor control unit in communication with the infusion pump and in communication with the signal receiving input for processing the digital pressure signals; and
f. a display in communication with the microprocessor for displaying parameters and processed pressure signals.

41. The console of claim 40 further comprising a user interface in communication with the microprocessor control unit for setting parameters.

42. The console of claim 40 further comprising the infusion pump being coupleable to the catheter so as to be in fluid communication with the catheter for the infusion of fluids through the catheter.

43. The console of claim 40 further comprising the signal receiving input being capable of receiving signals generated by a pressure transducer.

44. The console of claim 43 wherein the signals received by the signal receiving input are electrical signals.

45. The console of claim 40 wherein the infusion pump, signal receiving input, microprocessor, user interface and display are attached to a mounting platform.

46. The console of claim 45 wherein the mounting platform is a housing.

47. A console for use with an indwelling pressure monitoring, infusing and fluid collection catheter, the catheter having a pressure tube, an outer tube defining a lumen and hollow fibers in fluid communication with the lumen of the outer tube comprising:
a. an infusion pump coupleable to the catheter for infusion of fluids collected by the hollow fibers;
b. a signal receiving input in communication with a pressure signal conditioner in communication with a pressure transducer coupled to the pressure tube for converting analog pressure signals generated by the pressure transducer to digital pressure signals;
d. a microprocessor control unit in communication with the infusion pump for controlling fluid infusion according to parameters and processing received pressure signals;
e. a user interface in communication with the microprocessor control unit for setting parameters; and
f. a display for displaying set parameters and processed pressure signals.

48. The console of claim 46 further comprising the infusion pump being coupleable to the pressure tube of the catheter.

49. The console of claim 46 wherein the infusion pump, signal receiving input, microprocessor, user interface and display are attached to a mounting platform.

50. The console of claim 49 wherein the mounting platform is a housing.
